(19) United States
(12) Patent Application Publication
(10) Pub. No.: US 2021/0290208 A1
(43) Pub. Date: Sep. 23, 2021

(54) STOOL SAMPLING CONTAINER

(71) Applicant: FUJIFILM CORPORATION, Tokyo (JP)

(72) Inventor: Kaku IRISAWA, Kanagawa (JP)

(21) Appl. No.: 17/190,443

(22) Filed: Mar. 3, 2021

(30) Foreign Application Priority Data
Mar. 23, 2020 (JP) 2020-051708

(51) Int. Cl. A61B 10/00; B01L 3/00

(52) U.S. Cl. CPC A61B 10/0096; B01L 3/5029; A61B 10/0038

(57) ABSTRACT

In a container main body of the stool sampling container, in a case where a direction orthogonal to the longitudinal direction is defined as the width direction, the second surface opposed to a first surface into which a stool sampling rod is inserted and into which the nozzle is inserted has a nozzle insertion portion that allows an insertion of a nozzle at a position shifted in the width direction with respect to a coaxial position on an extension line of an axis of the stool sampling rod, and the insertion of the nozzle is regulated at a place other than the nozzle insertion portion.

### CROSS-REFERENCE TO RELATED APPLICATIONS

The present application claims priority under 35 U.S.C. § 119 to Japanese Patent Application No. 2020-051708, filed on Mar. 23, 2020. The above application is hereby expressly incorporated by reference, in its entirety, into the present application.

### BACKGROUND

### 1. Technical Field

The technology of the present disclosure relates to a stool sampling container.

### 2. Description of the Related Art

A stool sampling container used for a fecal occult blood test for detecting colorectal cancer is known. JP2006-029825A discloses a stool sampling container comprising a stool sampling rod for sampling stool and a container main body to which the stool sampling rod is attached. The container main body contains a storage solution in which the stool sampled by the stool sampling rod is suspended.

The stool sampling container disclosed in JP2006-029825A has a substantially rectangular planar shape. In the stool sampling container, the stool sampling rod is inserted from the first surface being located on one side in the longitudinal direction of the container, and a nozzle for extracting a suspension is inserted from the second surface opposed to the first surface in the longitudinal direction.

### SUMMARY

The stool sampling container is required to be miniaturized from the viewpoints of ease of mailing and portability. In the stool sampling container disclosed in JP2006-029825A, the nozzle is inserted from the second surface opposed to the first surface into which the stool sampling rod is inserted, and an insertion position of the nozzle is a coaxial position on the extension line of the axis of the stool sampling rod. Therefore, it is necessary to secure a space between a distal end of the nozzle and the stool sampling rod such that the distal end of the nozzle and the stool sampling rod do not come into contact with each other in a case where the nozzle is inserted, and it is difficult to shorten a length of the stool sampling container in the longitudinal direction.

An object of the technology of the present disclosure is to provide a stool sampling container having a small size in the longitudinal direction.

A stool sampling container of the present disclosure comprises a stool sampling rod that samples stool, and a container main body to which the stool sampling rod is attached and that contains a storage solution in which the stool sampled by the stool sampling rod is suspended, the container main body having a first surface provided at one end in a longitudinal direction parallel to an axial direction of the stool sampling rod in an attached state, and a second surface provided at the other end in the longitudinal direction and opposed to the first surface, in which the stool sampling rod is inserted from the first surface, and a nozzle for extracting the storage solution is inserted from the second surface in parallel to the stool sampling rod, in which in a case where a direction orthogonal to the longitudinal direction is defined as a width direction, the second surface has a nozzle insertion portion that allows an insertion of the nozzle at a position shifted in the width direction with respect to a coaxial position on an extension line of an axis of the stool sampling rod, and regulates the insertion of the nozzle at a place other than the nozzle insertion portion.

It is preferable that the nozzle insertion portion has a first nozzle insertion portion into which the nozzle is inserted in a case of extracting the storage solution and a second nozzle insertion portion into which the nozzle is inserted in a case of forming an air hole with respect to the second surface at a position different from the first nozzle insertion portion.

It is preferable that the first nozzle insertion portion and the second nozzle insertion portion are disposed on both sides about the stool sampling rod in the width direction.

It is preferable that the container main body has a symmetrical shape with the stool sampling rod as a center axis in the width direction.

It is preferable that the stool sampling container further comprises a storage space that is partitioned by a partition wall inside the container main body and is capable of storing the storage solution in the partition wall, and into which a part including a distal end of the stool sampling rod and a part including a distal end of the nozzle are inserted, in which the storage space has a wide portion formed at an end portion on the second surface side and having a width wider than that of the first surface side, and the wide portion has a width which allows the part of the nozzle inserted from the nozzle insertion portion and the part of the stool sampling rod to overlap each other in the longitudinal direction.

According to the technique of the present disclosure, it is possible to provide a small size of the stool sampling container.

### BRIEF DESCRIPTION OF THE DRAWINGS

Exemplary embodiments according to the technique of the present disclosure will be described in detail based on the following figures, wherein:

FIG. 1 is a perspective view of a stool sampling container,

FIG. 2 is an exploded perspective view of a stool sampling container,

FIG. 3 is view showing a periphery of the storage space. (A) of FIG. 3 shows a plan view of a container main body viewed from the second surface side, and (B) of FIG. 3 shows a cross-sectional view of the container main body, which is cut along the longitudinal direction,

FIG. 4 is a diagram showing a state in which stool is sampled with a stool sampling rod,

FIG. 5 is a diagram showing a state in which a stool sampling rod in which stool is captured by a capturing portion is returned to a container main body,

FIG. 6 is diagram showing handling of a stool sampling container in a test apparatus. (A) of FIG. 6 shows a state in which a nozzle is inserted into a storage space in a case of forming an air hole in a seal, and (B) of FIG. 6 shows a state in which a nozzle is inserted into a storage space in a case of extracting a suspension,

FIG. 7 is view showing a periphery of the storage space. (A) of FIG. 7 shows a plan view of a container main body viewed from the second surface side, and (B) of FIG. 7 shows a cross-sectional view of the container main body, which is cut along the longitudinal direction, and

FIG. 8 is diagram showing handling of a stool sampling container in a test apparatus. (A) of FIG. 8 shows a state in which a nozzle is inserted into a storage space in a case of forming an air hole in a seal, and (B) of FIG. 8 shows a state in which a nozzle is inserted into a storage space in a case of extracting a suspension.

### DETAILED DESCRIPTION

### First Embodiment

Configuration of Stool Sampling Container

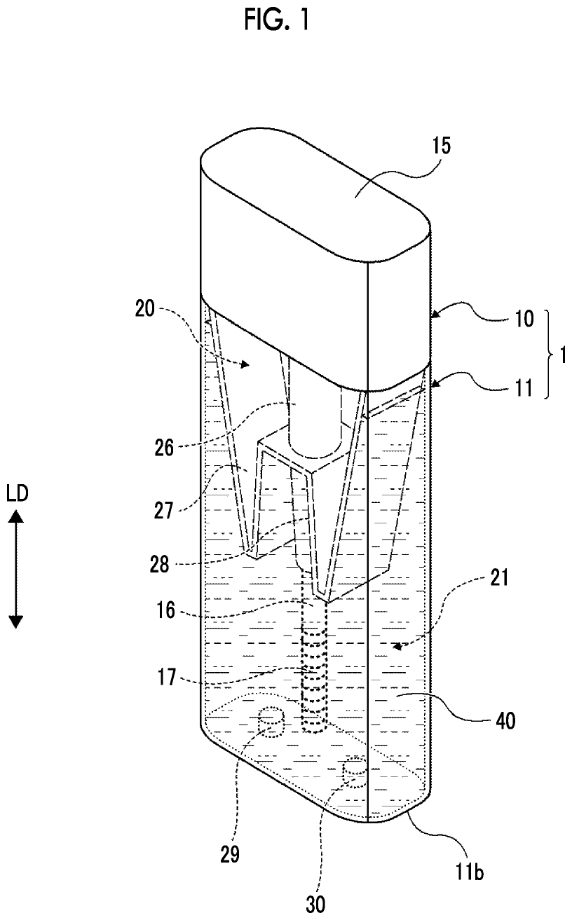

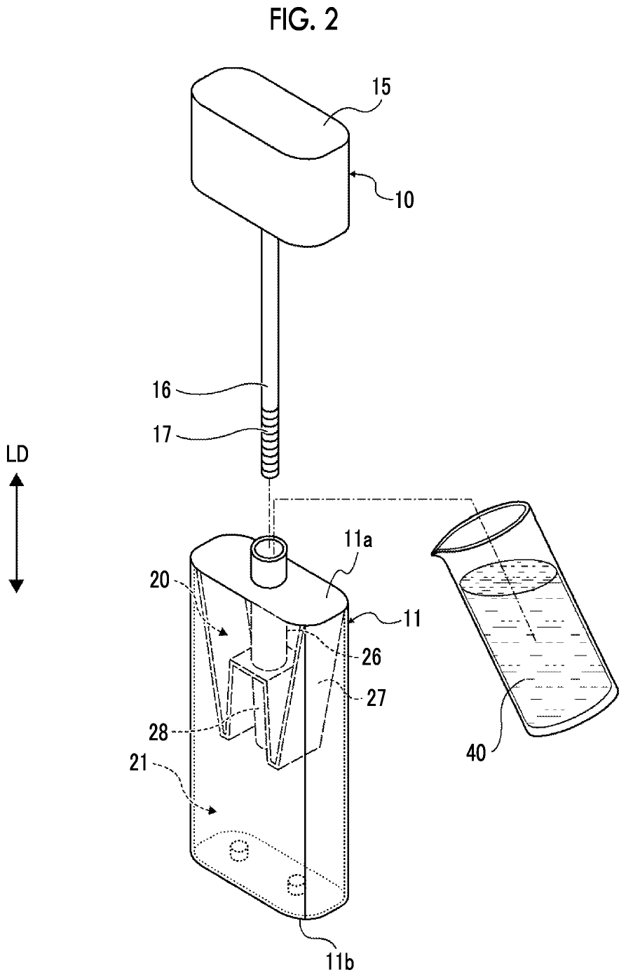

FIG. 1 is a perspective view of a stool sampling container 1 according to the first embodiment of the present disclosure. FIG. 2 is an exploded perspective view of the stool sampling container 1. In the following description, for the sake of convenience, the upper side in FIG. 1 and FIG. 2 will be described as the first surface side of the stool sampling container 1, and the lower side in FIG. 1 and FIG. 2 will be described as the second surface side of the stool sampling container 1.

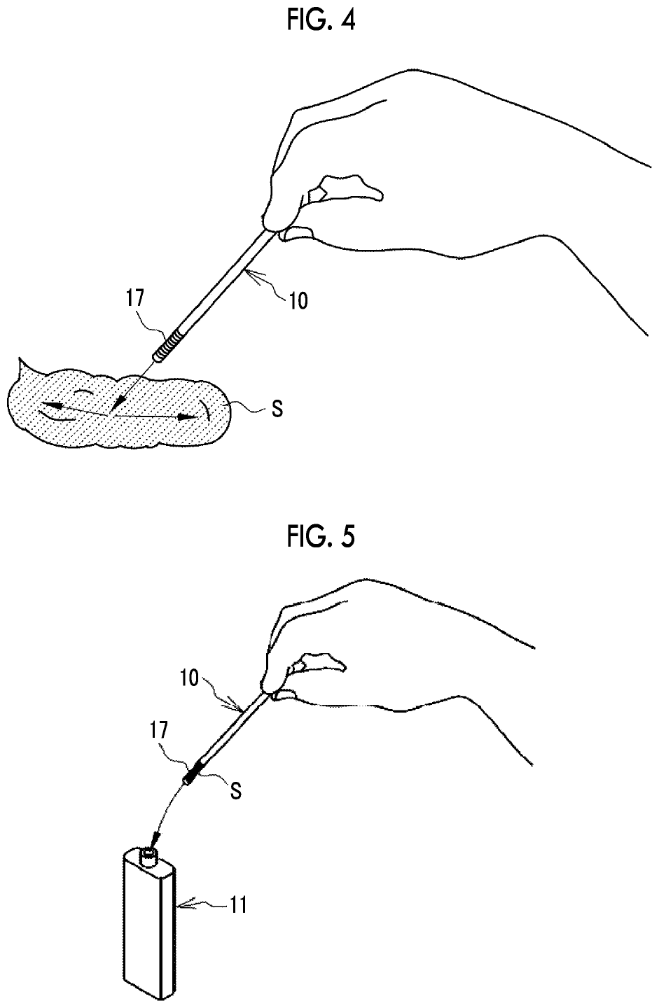

In FIG. 1 and FIG. 2, the stool sampling container 1 is used as an example for a fecal occult blood test for detecting colorectal cancer. The stool sampling container 1 has a substantially rectangular parallelepiped shape that is elongated and flat as a whole. The stool sampling container 1 comprises a stool sampling rod 10 for sampling stool S (refer to FIG. 4 and the like) and a container main body 11 to which the stool sampling rod 10 is attachably and detachably attached.

The stool sampling rod 10 is formed of a colored resin as an example. The stool sampling rod 10 is configured with a cap 15, a rod main body 16, and a capturing portion 17. The cap 15 has a substantially rectangular parallelepiped shape and is gripped by a subject at the time of sampling stool (refer to FIG. 4 and the like). The rod main body 16 has an elongated cylindrical shape extending downward from the cap 15. The capturing portion 17 is formed at a distal end of the rod main body 16. A plurality of grooves for efficiently capturing the stool S are formed in the capturing portion 17.

The container main body 11 is formed of a transparent resin such that the inside can be visually recognized from the outside. The transparent resin includes, for example, an acrylonitrile-butadiene-styrene (ABS) resin, polyethylene, polypropylene, and the like.

The container main body 11 is attached with the stool sampling rod 10 and stores a storage solution 40 in which the stool sampled by the stool sampling rod 10 is suspended. The stool sampling rod 10 is inserted into the container main body 11 in a posture in which the axial direction is parallel to a longitudinal direction LD. In the container main body 11, one end in the longitudinal direction LD parallel to the axial direction (the axial direction of the rod main body 16 in the first embodiment) of the attached stool sampling rod 10 has the first surface 11a, and the other end has the second surface 11b opposed to the first surface 11a. From the first surface 11a, the stool sampling rod 10 is inserted, and from the second surface 11b, as described later, a nozzle 60 for extracting the storage solution 40 is inserted.

A cylindrical shaped plug portion 25 which is press-fitted to the cap 15 to prevent liquid leakage from the container main body 11 is formed on the first surface 11a (refer to FIG. 2) of the container main body 11. The plug portion 25 functions as a stool sampling rod insertion portion.

The container main body 11 is partitioned by a partition wall 27 inside an outer peripheral wall 11c, and has a storage space 21 capable of storing the storage solution 40. Between the outer peripheral wall 11c and the storage space 21, there is a buffer space 20 for relaxing an impact and the like from the outside.

In the buffer space 20, an insertion portion 26 is provided between the first surface 11a and the storage space 21. The insertion portion 26 is a tubular body having a cylindrical shape. In the insertion portion 26, in a case where the stool sampling rod 10 is inserted from the plug portion 25 of the first surface 11a into the container main body 11, the rod main body 16 including the capturing portion 17 which has passed through the plug portion 25 is inserted.

On the second surface 11b side of the insertion portion 26, a scraping-off portion 28 for scraping off excess stool adhering to the capturing portion 17 is provided. The scraping-off portion 28 is a conical tubular body. The inner diameter of the scraping-off portion 28 is gradually narrowed from the insertion portion 26 toward the second surface 11b side to the same extent as the outer diameter of the capturing portion 17. The scraping-off portion 28 is integrally molded with, for example, the partition wall 27.

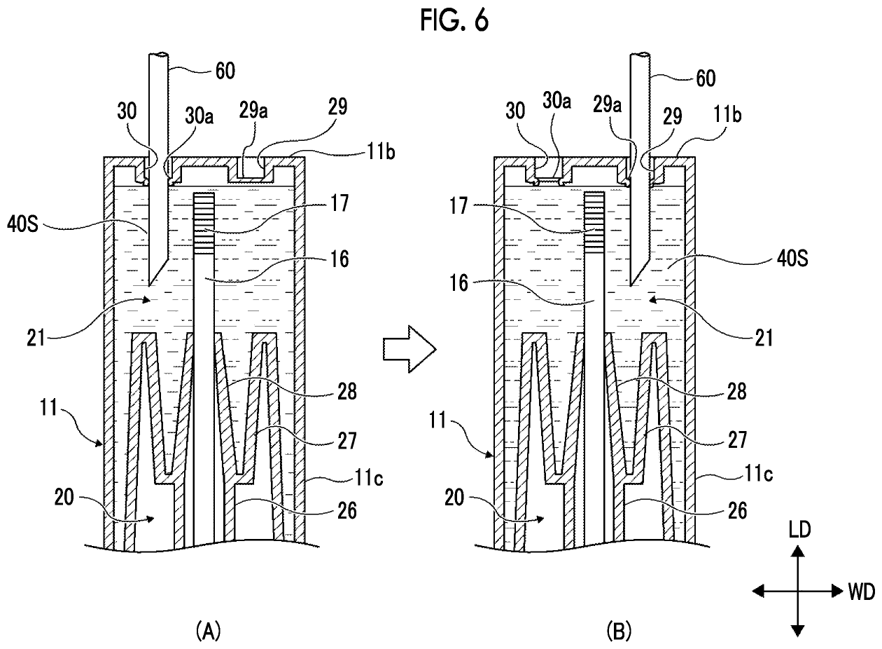

The storage solution 40 is stored in the storage space 21. The storage solution 40 contains water as the main component. In the state shown in FIG. 1 in which the stool sampling rod 10 is attached to the container main body 11, the entire capturing portion 17 enters the storage space 21, and the distal end of the capturing portion 17 reaches a vicinity of the second surface 11b in the longitudinal direction LD of the container main body 11. That is, a part including the capturing portion 17 which is the distal end of the stool sampling rod 10 is inserted into the storage space 21. Then, about 90% of the storage space 21 is filled with the storage solution 40. Therefore, the entire capturing portion 17 is immersed in the storage solution 40. The stool S captured by the capturing portion 17 is suspended in the storage solution 40. Hereinafter, the storage solution 40 in which the stool S is suspended is referred to as a suspension 40S (refer to FIG. 6).

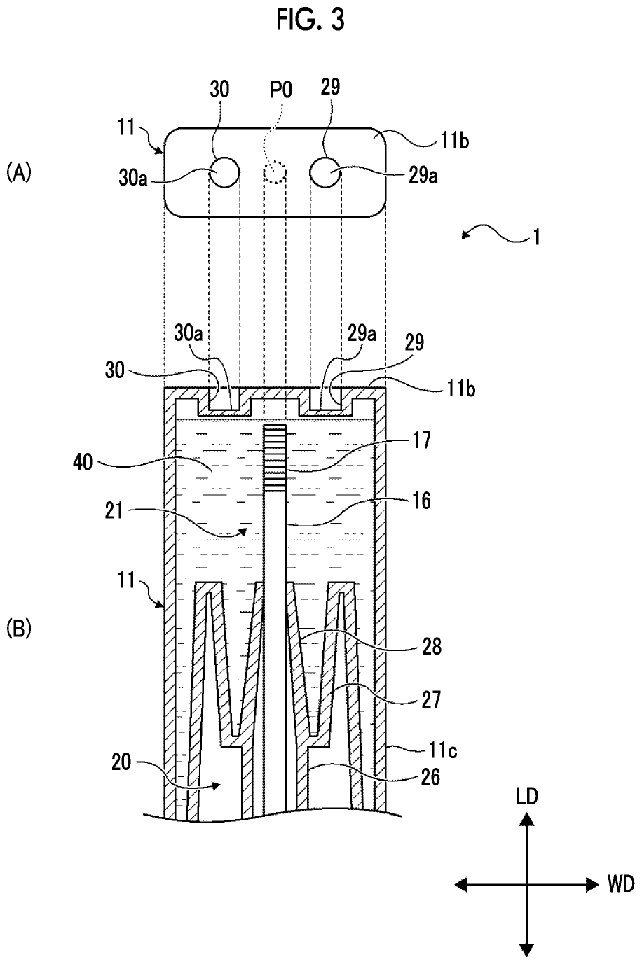

FIG. 3 is view showing a periphery of the storage space 21. (A) of FIG. 3 shows a plan view of a container main body 11 viewed from the second surface 11b side, and (B) of FIG. 3 shows a cross-sectional view of the container main body 11, which along the longitudinal direction. It should be noted that (B) of FIG. 3 shows a state in which the container main body 11 is turned upside down from that in FIG. 1. That is, in (B) of FIG. 3, the second surface 11b faces upward. In addition, in (B) of FIG. 3, the rod main body 16 of the stool sampling rod 10 is shown not as a cross-sectional view but as a side view. Further, as shown in (A) of FIG. 3 and (B) of FIG. 3, in the container main body 11, a direction orthogonal to the longitudinal direction LD is defined as a width direction WD.

As shown in (A) of FIG. 3 and (B) of FIG. 3, the second surface 11b of the container main body 11 has, at a position shifted in the width direction WD with respect to a coaxial position PO on the extension line of the axis (the rod main body 16 in the first embodiment) of the stool sampling rod 10, the first nozzle insertion portion 29 into which the nozzle 60 is inserted in a case of extracting the suspension 40S. In addition, the second nozzle insertion portion 30 into which the nozzle is inserted in a case of forming an air hole with respect to the storage space 21 is provided at a position different from the first nozzle insertion portion 29. The coaxial position PO is a position at which the second surface 11b and the extension line of the axis of the stool sampling rod 10 intersect.

The first nozzle insertion portion 29 is a concave portion that is integrally molded with the container main body 11 on the second surface 11b of the container main body 11 and is concave inside the container main body 11. A bottom surface 29a of the concave portion is a thin portion that is relatively thinner than the outer peripheral wall 11c. The thickness of the thin portion is a thickness which can be pierced by the nozzle 60.

Similarly, the second nozzle insertion portion 30 is a concave portion that is integrally molded with the container main body 11 on the second surface 11b of the container main body 11 and is concave inside the container main body 11. A bottom surface 30a of the concave portion is a thin portion that is relatively thinner than the outer peripheral wall 11c. The thickness of the thin portion is a thickness which can be pierced by the nozzle 60.

On the second surface 11b of the container main body 11, places other than the bottom surface 29a of the first nozzle insertion portion 29 and the bottom surface 30a of the second nozzle insertion portion 30 are formed in a thickness which can not be pierced by the nozzle 60. Therefore, the insertion of the nozzle 60 is regulated at places other than the first nozzle insertion portion 29 and the second nozzle insertion portion 30.

The first nozzle insertion portion 29 and the second nozzle insertion portion 30 are disposed on both sides about the rod main body 16 of the stool sampling rod 10 in the width direction WD. In addition, the container main body 11 has a symmetrical shape with the rod main body 16 the stool sampling rod 10 as a center axis in the width direction WD.

Procedure of Examination

Next, a procedure from stool sampling by the stool sampling container 1 having the above configuration to the fecal occult blood test will be described with reference to FIG. 4 to FIG. 6. First, a subject excretes stool S into a toilet bowl. Then, the stool sampling rod 10 is removed from the container main body 11, and as shown in FIG. 4, the capturing portion 17 of the stool sampling rod 10 is brought into contact with the stool S, and the capturing portion 17 traces a surface of the stool S. Since the capturing portion 17 has a plurality of grooves, it is possible to easily capture a necessary and sufficient amount of stool S for the fecal occult blood test.

Subsequently, as shown in FIG. 5, the subject returns the stool sampling rod 10 in which the stool S is captured by the capturing portion 17 to the container main body 11 and puts it in the state shown in FIG. 1. At this time, the capturing portion 17 is inserted through the insertion portion 26, passes through the scraping-off portion 28, and reaches the storage space 21. In a case where the capturing portion 17 passes through the scraping-off portion 28, the excess stool S among the stools S captured by the capturing portion 17 is removed.

After stool sampling, the stool sampling container 1 is transported to a test facility where the fecal occult blood test is performed. During this transportation, the storage solution 40 in the storage space 21 is agitated. Then, the stool S captured by the capturing portion 17 is diffused into the storage solution 40, and the suspension 40S suitable for a fecal occult blood test is generated.

As shown in FIG. 6, in the test facility, the stool sampling container 1 is set on a mounting portion of the test apparatus (not shown) in a state of being upside down from that in FIG. 1. Then, in the test apparatus, the nozzle 60 pierces into the first nozzle insertion portion 29 and the second nozzle insertion portion 30, and is inserted into the storage space 21 of the container main body 11. In the test apparatus, the positional relationship between the nozzle 60 and the mounting portion is determined. The nozzle 60 is configured to be movable between a position at which the nozzle 60 is inserted into the first nozzle insertion portion 29 and a position at which the nozzle 60 is inserted into the second nozzle insertion portion 30 on the second surface 11b of the container main body 11 mounted on the mounting portion.

In the test apparatus, first, the nozzle 60 is inserted into the storage space 21 from the second nozzle insertion portion 30, and an air hole for the storage space 21 is formed. Next, after the nozzle 60 is inserted into the storage space 21 from the first nozzle insertion portion 29, the suspension 40S in the storage space 21 is extracted by the nozzle 60 for the fecal occult blood test.

Action Effect

As described above, the second surface 11b of the container main body 11 has, at a position shifted in the width direction WD with respect to a coaxial position PO on the extension line of the axis of the stool sampling rod 10, the nozzle insertion portions into which the nozzle 60 is inserted. In addition, the insertion of the nozzle 60 is regulated at a place other than the nozzle insertion portions.

As described above, in the stool sampling container 1 of the present disclosure, in a case where the nozzle 60 is inserted, since the nozzle 60 and the stool sampling rod 10 are overlapped each other in the longitudinal direction LD in the storage space 21, in comparison with a conventional stool sampling container in which the nozzle is inserted at a coaxial position with the axis of the stool sampling rod, the lengths of the storage space 21 and the container main body 11 having the storage space 21 inside can be reduced in the longitudinal direction LD. That is, in the prior art, since the stool sampling rod and the nozzle are at a coaxial position in the storage space, the length of the storage space in the longitudinal direction LD is required to be long enough to secure a space for disposing the nozzle on the extension line of the stool sampling rod. On the other hand, in the stool sampling container 1 of the present disclosure, since the nozzle 60 is inserted at a position shifted in the width direction WD with respect to the axial direction of the stool sampling rod 10, the lengths of the storage space 21 and the container main body 11 can be shortened in the longitudinal direction LD.

In addition, the nozzle insertion portions has the first nozzle insertion portion 29 into which the nozzle 60 is inserted in a case of extracting the suspension 40S and the second nozzle insertion portion 30 into which the nozzle 60 is inserted at a position different from the first nozzle insertion portion 29 in a case of forming an air hole with respect to the storage space 21. Therefore, since the air hole can be formed at a position different from the position where the nozzle 60 is inserted in a case of extracting the suspension 40S, the inside of the storage space 21 does not have negative pressure in a case of extracting the suspension 40S, and the suspension 40S can be stably extracted.

In addition, the first nozzle insertion portion 29 and the second nozzle insertion portion 30 are disposed on both sides about the rod main body 16 of the stool sampling rod 10 in the width direction WD. Therefore, in the case where the nozzle 60 can be inserted into two different positions as described above, it is not necessary to widen the width from the rod main body 16 to one side, so that the stool sampling container 1 can be easily miniaturized.

In addition, the container main body 11 has a symmetrical shape with the rod main body 16 the stool sampling rod 10 as a center axis in the width direction WD. Therefore, in a case where the stool sampling container 1 is mounted into the test apparatus in which the nozzle 60 is inserted into two different positions as described above, the stool sampling container 1 can be mounted without worrying horizontal directions, so that convenience can be improved.

### Second Embodiment

A stool sampling container 2 of the second embodiment is formed by changing the shape of the buffer space 20 and the storage space 21 from the stool sampling container 1 of the first embodiment. Therefore, the description of the same portion as that of the stool sampling container 1 of the first embodiment will be omitted.

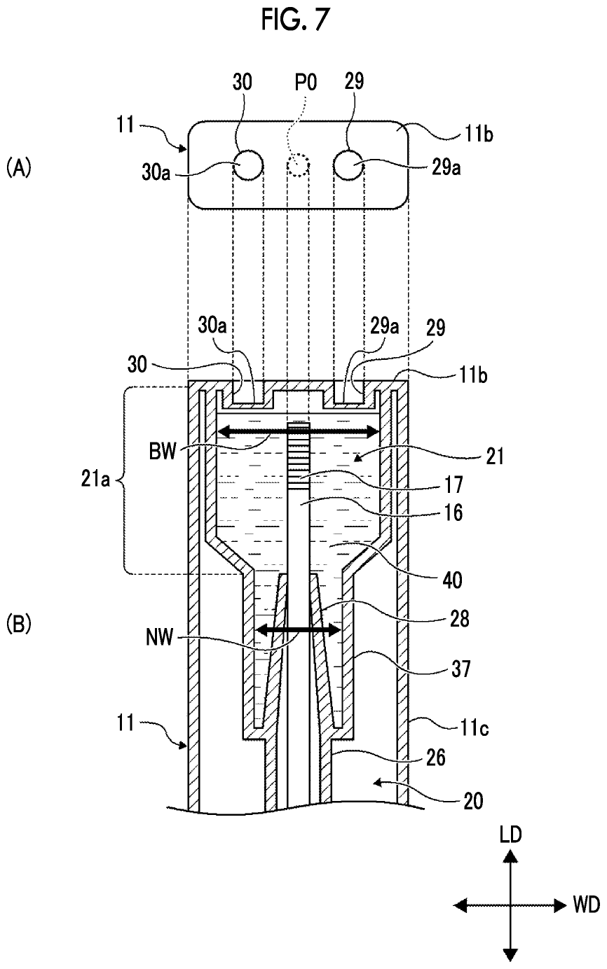

As shown in (A) of FIG. 7 and (B) of FIG. 7, the container main body 11 is partitioned by a partition wall 37 inside an outer peripheral wall 11c, and has a storage space 21 capable of storing the storage solution 40. Between the outer peripheral wall 11c and the storage space 21, there is a buffer space 20 for relaxing an impact and the like from the outside.

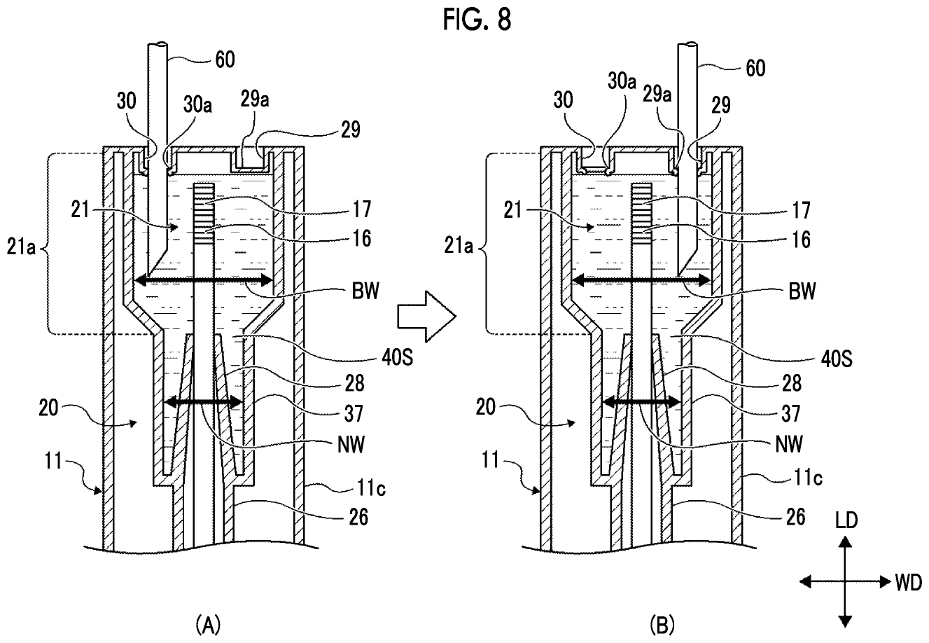

As shown in (A) of FIG. 8 and (B) of FIG. 8, in the width direction WD, the storage space 21 has a wide portion 21a having a width wider than that of the first surface 11a side at the end portion on the second surface 11b side. A width BW of the wide portion 21a is an interval between the partition walls 37 in the width direction WD. In the storage space 21, an interval between the partition walls 37 on a side closer to the first surface 11a than the wide portion 21a is a width NW. The width BW is wider than the width NW. The wide portion 21a can receive the nozzle 60 inserted from the first nozzle insertion portion 29 and the second nozzle insertion portion 30 shifted in the width direction WD with respect to a coaxial position PO on an extension line of the axis of the stool sampling rod 10, and has the width BW allowing parts of the nozzle 60 and the stool sampling rod 10 to overlap each other in the longitudinal direction LD. The coaxial position PO is a position at which the second surface 11b and the extension line of the axis of the stool sampling rod 10 intersect.

The wide portion 21a extends to both sides about the rod main body 16 of the stool sampling rod 10 in the width direction WD. In addition, the container main body 11 and the storage space 21 have a symmetrical shape with the rod main body 16 of the stool sampling rod 10 as a center axis in the width direction WD.

As described above, in the storage space 21, only the width BW of the wide portion 21a into which the nozzle 60 is inserted is widened, and the width NW of a region other than the wide portion 21a remains narrower than the width BW, so that the increase in the amount of the storage solution 40 stored in the storage space 21 can be suppressed.

### Modification Example

The technology of the present disclosure can be appropriately combined with the above-described various embodiments and various modification examples.

For example, as the nozzle insertion portion, only the first nozzle insertion portion 29 into which the nozzle 60 is inserted in a case of extracting the suspension 40S may be provided instead of providing two nozzle insertion portions, which are the first nozzle insertion portion 29 into which the nozzle 60 is inserted in a case of extracting the suspension 40S and the second nozzle insertion portion 30 into which the nozzle 60 is inserted in a case of forming an air hole with respect to the second surface 11b as described in the above embodiment.

In addition, the technology of the present disclosure is not limited to the above-described embodiments, and it is needless to say that various configurations can be adopted without departing from the gist of the present invention.

The description content and the illustrated content described above are detailed descriptions of portions related to the technology of the present disclosure, and are merely an example of the technology of the present disclosure. For example, the above description of the configurations, functions, actions, and effects is an example of the configurations, functions, actions, and effects of the portions according to the technology of the present disclosure. Therefore, it is needless to say that in the description content and the illustrated content described above, unnecessary portions may be deleted and new elements may be added to or replaced within the scope not deviating from the gist of the technology of the present disclosure. In addition, in order to avoid confusion and to facilitate understanding portions according to the technology of the present disclosure, the description of the common general technical knowledge and the like, which need not be specifically explained in order to enable implementation of the technology of the present disclosure, is omitted from the description content and the illustrated content described above.

All of the documents, patent applications, and technical standards described herein are incorporated herein by reference to the same extent as a case where the individual documents, patent applications, and technical standards are specifically and individually indicated to be incorporated by reference.

What is claimed is:

1. A stool sampling container comprising:
a stool sampling rod that samples stool; and
a container main body to which the stool sampling rod is attached and that contains a storage solution in which the stool sampled by the stool sampling rod is suspended, the container main body having a first surface provided at one end in a longitudinal direction parallel to an axial direction of the stool sampling rod in an attached state, and a second surface provided at the other end in the longitudinal direction and opposed to the first surface, in which the stool sampling rod is inserted from the first surface, and a nozzle for extracting the storage solution is inserted from the second surface in parallel to the stool sampling rod,
wherein in a case where a direction orthogonal to the longitudinal direction is defined as a width direction, the second surface has a nozzle insertion portion that allows an insertion of the nozzle at a position shifted in the width direction with respect to a coaxial position on an extension line of an axis of the stool sampling rod, and regulates the insertion of the nozzle at a place other than the nozzle insertion portion.

2. The stool sampling container according to claim 1,
wherein the nozzle insertion portion has a first nozzle insertion portion into which the nozzle is inserted in a case of extracting the storage solution and a second nozzle insertion portion into which the nozzle is inserted in a case of forming an air hole with respect to the second surface at a position different from the first nozzle insertion portion.

3. The stool sampling container according to claim 2,
wherein the first nozzle insertion portion and the second nozzle insertion portion are disposed on both sides about the stool sampling rod in the width direction.

4. The stool sampling container according to claim 3,
wherein the container main body has a symmetrical shape with the stool sampling rod as a center axis in the width direction.

5. The stool sampling container according to claim 1, further comprising:
a storage space that is partitioned by a partition wall inside the container main body and is capable of storing the storage solution in the partition wall, and into which a part including a distal end of the stool sampling rod and a part including a distal end of the nozzle are inserted,
wherein the storage space has a wide portion formed at an end portion on the second surface side and having a width wider than that of the first surface side, and
the wide portion has a width which allows the part of the nozzle inserted from the nozzle insertion portion and the part of the stool sampling rod to overlap each other in the longitudinal direction.
